(19) United States
(12) Plant Patent Application Publication
(10) Pub. No.: US 2011/0197326 P1
(43) Pub. Date: Aug. 11, 2011

(54) Dianella caerulea x brevipedunculata plant named 'Indigo Bells'

(50) ;

(76) Inventors: Charles Mines, Park Ridge (AU); Francis Benson, Park Ridge (AU)

(21) Appl. No.: 12/658,420

(22) Filed: Feb. 11, 2010

(51) Int. Cl. A01H 5/00

(52) U.S. Cl. PLT/424

(57) ABSTRACT

‘Indigo Bells’ is a distinctive variety of Dianella caerulea which is characterized by the combination of its short to medium plant height, dense upright growth habit with flowers positioned just above the foliage, upright leaf attitude with distal weeping, year round flowering season, and violet blue flower color with violet blue berry color.

### LATIN NAME OF THE GENUS AND SPECIES

The Latin name of the novel variety disclosed herein is Dianella caerulea×brevipedunculata.

### VARIETY DENOMINATION

The inventive variety of Dianella caerulea×brevipedunculata disclosed herein has been given the varietal denomination ‘Indigo Bells’.

### BACKGROUND OF THE INVENTION

The present invention relates to a new and distinct perennial Blue Flax Lilly variety of Dianella caerulea×brevipedunculata, which has been given the varietal denomination of ‘Indigo Bells’. Its market class is that of an ornamental grass-like plant. ‘Indigo Bells’ is intended for use in landscaping and as a decorative grass-like plant.

An application for plant breeders' rights for variety ‘Indigo Bells’ has been filed with the Australian Plant Breeders' Rights Office, under the name ‘Weeping Kate’ on 9 Jun. 2009 under Application No. 2009/138.

Parentage. The Dianella caerulea×brevipedunculata variety ‘Indigo Bells’ was first discovered in 2005 in an Australian nursery in Park Ridge, Queensland, Australia as part of a seed production program. ‘Indigo Bells’ is a result of an interspecific cross from open pollinated Dianella caerulea grown alongside Dianella brevipedunculata. The ‘Indigo Bells’ maternal parent, Dianella caerulea is characterized by a short to medium plant height (to 50 cm), flowering stem extending well above foliage and a typical seasonal spring to summer flowering period. The ‘Indigo Bells’ pollen parent, Dianella brevipedunculata is characterized by a short to medium plant height (to 50 cm), flowering stem position well below the foliage and a typical seasonal spring to summer flowering period.

In 2004, Dianella caerulea and Dianella brevipedunculata. were grown in separate adjacent open beds for the purpose of seed collection. The plants were open pollinated and each species type was collected separately for growing on to seedling stage.

Several thousand seedling plants were grown. In 2005 fifty plants were identified as having short plant heights. These plants were selected and grown to maturity for evaluation. A single plant from these was found to produce flowers from a small size. It was micropropagated and then planted out to test if the unique traits were reproduced over successive generation of vegetative reproduction. These plants were grown for 12 months and subsequently found to flower for 12 months of the year with a flowering stem presentation intermediate between that of Dianella caerulea and Dianella brevipedunculata. It was also found to have other characteristics of Dianella brevipedunculata including a clumping (not running) plant growth habit and resistance to root rotting that affects Dianella caerulea, but not Dianella brevipedunculata. It was subsequently named “Weeping Kate’ in Australia and given the name ‘Indigo Bells’ in the USA.

Asexual reproduction. ‘Indigo Bells’ was first asexually propagated by micropropagation in 2005, in the state of Queensland, Australia. It was asexually reproduced again from 2005 to 2009 and confirmed to be stable in character. The distinctive characteristics of the inventive variety, ‘Indigo Bells’, have remained stable and true to type from generation to generation through successive cycles of asexual reproduction including vegetative division and more than twenty generations in micropropagation.

### SUMMARY OF THE INVENTION

‘Indigo Bells’ is a distinctive variety of Dianella caerulea×brevipedunculata which is characterized by the combination of its short to medium plant height, dense upright growth habit with flowers positioned just above the foliage, upright leaf attitude with distal weeping, year round flowering season, violet blue flower color with violet blue berry color, which is unusual for a Dianella caerulea or Dianella brevipedunculata.

### BRIEF DESCRIPTION OF THE FIGURES

The photographs in the drawings were made using conventional techniques and show the colours as true as reasonably possible by conventional photography. Colours in the photographs may differ slightly from the colour values cited in the detailed botanical description, which accurately describe the colours of the new Dianella caerulea×brevipedunculata.

FIG. 1 shows an ‘Indigo Bells’ plant and illustrates the weeping leaf habit and flower position just above foliage that is characteristic of the variety.

FIG. 2 shows the stem and leaf basil sheath of ‘Indigo Bells’.

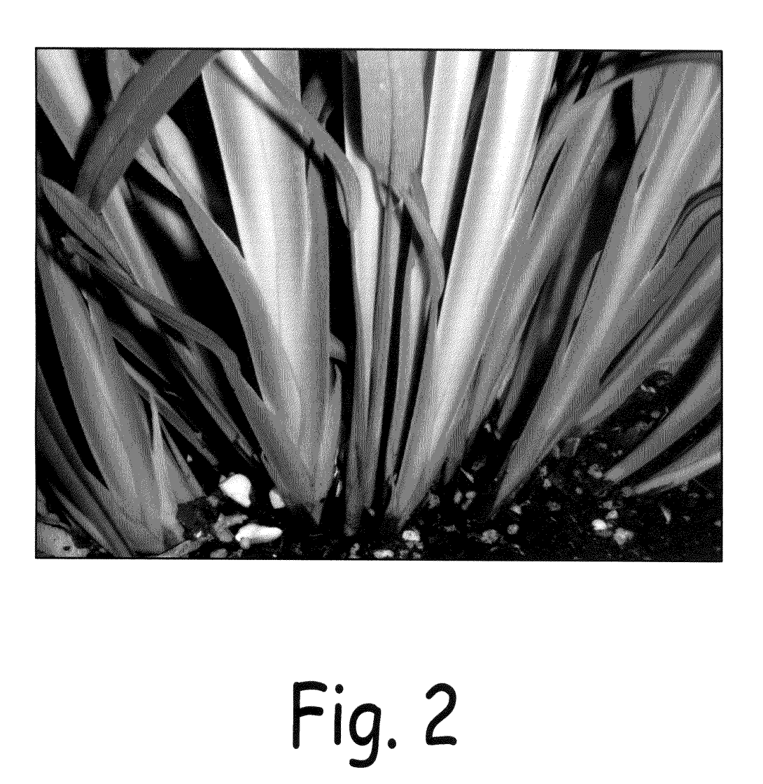

FIG. 3 shows the leaf of ‘Indigo Bells’.

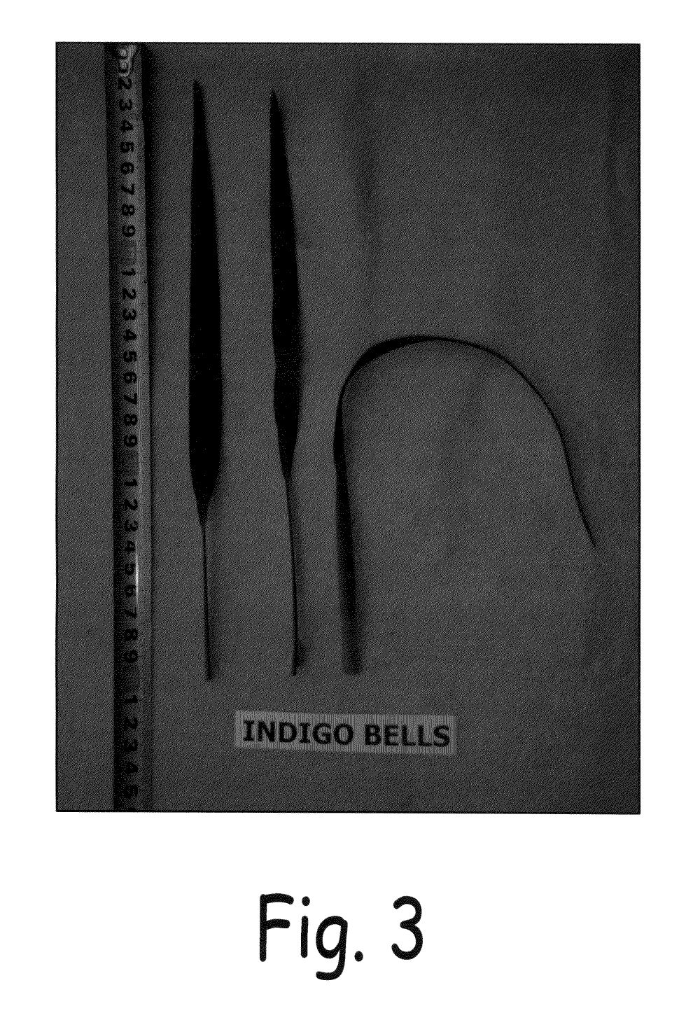

FIG. 4 shows the inflorescence of ‘Indigo Bells’.

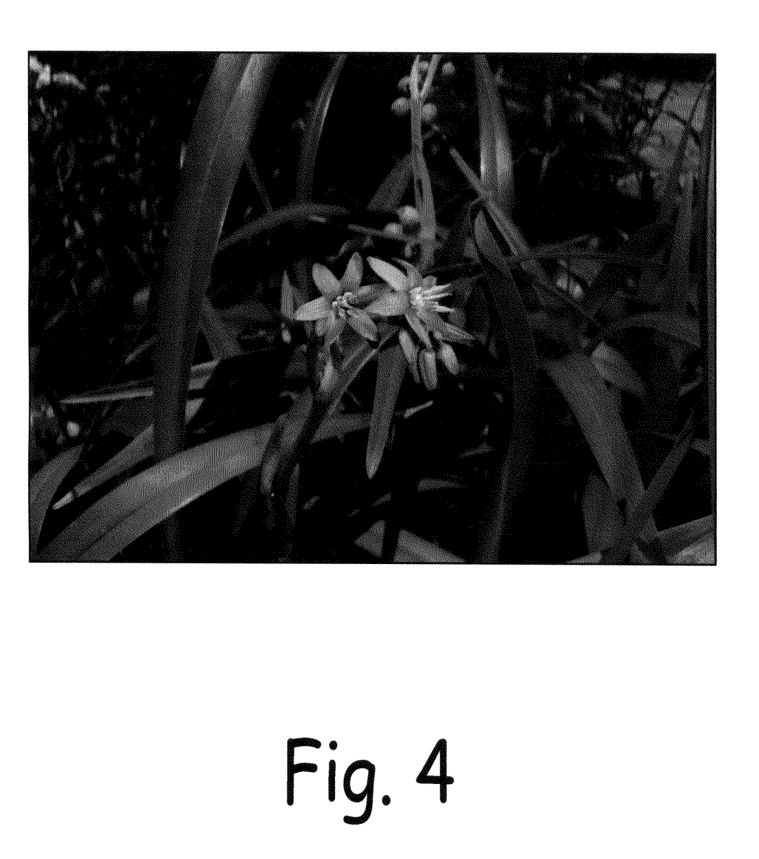

FIG. 5 shows the inflorescence position of ‘Indigo Bells’.

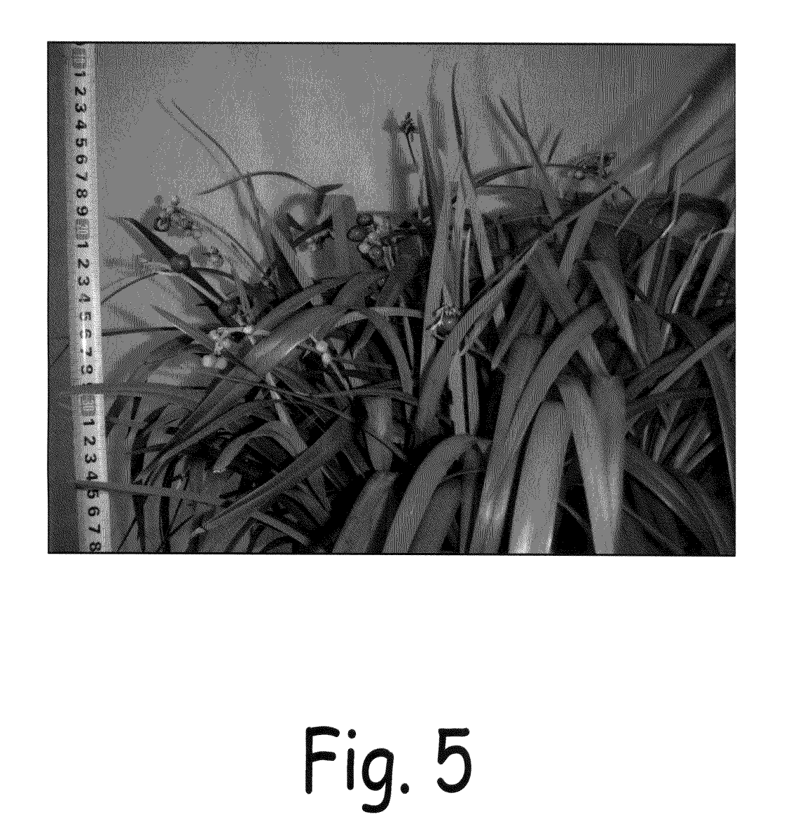

FIG. 6 shows the berry of ‘Indigo Bells’.

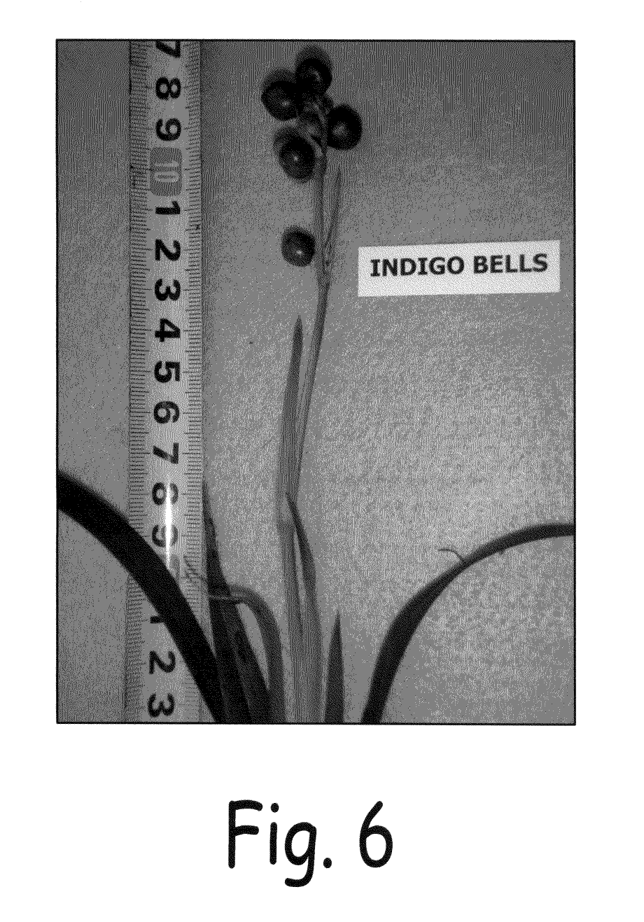

FIG. 7 shows the seed of ‘Indigo Bells’.

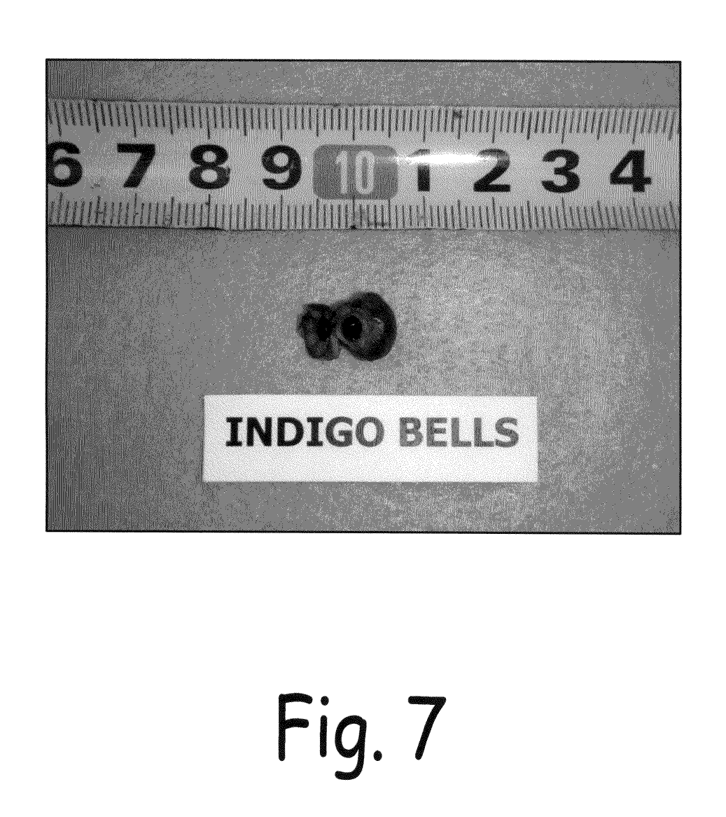

### DETAILED BOTANICAL DESCRIPTION OF THE PLANT

The following is a detailed botanical description of a new and distinct variety of a Dianella caerulea×brevipedunculata ornamental grass-like plant known as ‘Indigo Bells’. The descriptions disclosed herein are based upon observations of the plant grown in 250 mm nursery pots and field plots in Queensland, Australia. The plants were approximately 12-months-old at the time of observation. All colors cited herein refer to The Royal Horticultural Society Colour Chart (The Royal Horticultural Society, London, 2007 edition).

Those skilled in the art will appreciate that certain characteristics will vary with older or, conversely, younger plants. ‘Indigo Bells’ has not been observed under all possible environmental conditions. Where dimensions, sizes, colors and other characteristics are given, it is to be understood that such characteristics are approximations or averages set forth as accurately as practicable. The phenotype of the variety may differ with variations in the environment such as season, temperature, light intensity, day length, cultural conditions and the like.

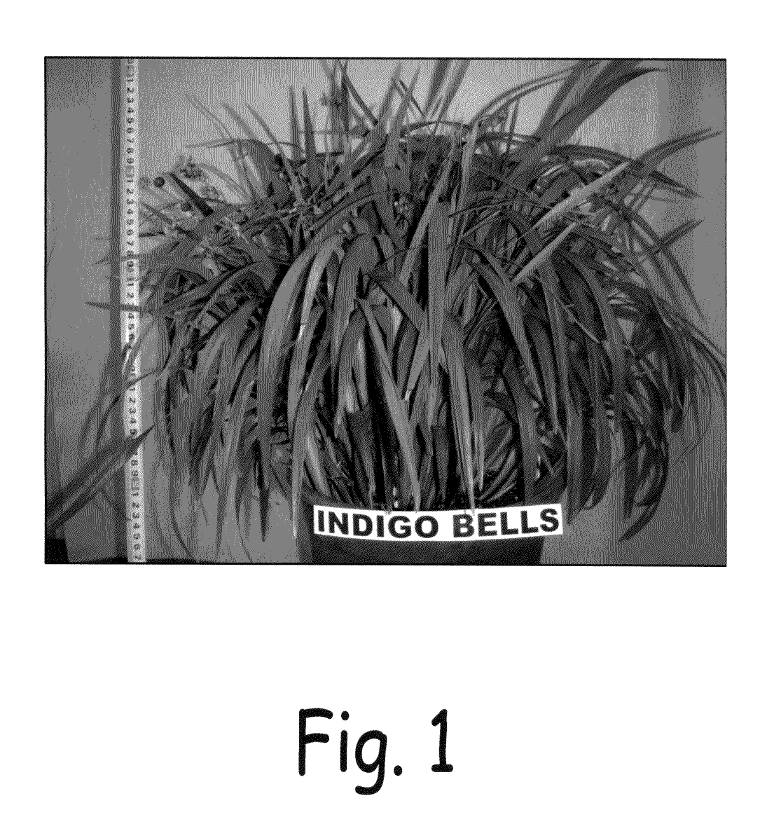

‘Indigo Bells’ is a perennial Dianella caerulea×brevipedunculata plant, which was produced through an innovative breeding program. After its selection, ‘Indigo Bells’ was asexually propagated by micropropagation. ‘Indigo Bells’ has dense growth, upright and compact growth habit, short rhizome form, violet blue flower colour, short internodes located on short canes, flowering stems bearing flowers positioned just above the foliage which subsequently produce violet blue berries. A botanical description of ‘Indigo Bells’ and a comparison with other of Dianella caerulea and Dianella brevipedunculata are provided below. A representative Dianella caerulea×brevipedunculata ‘Indigo Bells’ plant is shown in FIG. 1.

### Technical Description of the Variety

Growth habit: ‘Indigo Bells’ is a rhizomatous plant forming a semi-compact tussock. Its growth habit is erect, the plant height is short to medium, (mean 50 cm), plant width is medium (55 to 60 cm) and the density of shoots is strong.
Stem: Length of internodes is short (to 10 mm).
Foliage: Leaf blade attitude erect and weeping distally, length medium (23-29 cm), width medium (13-15 mm), upper side color green (RHS N137A), lower side colour yellow-green (RHS 147B to N137C), upper side glaucosity weak, upper side is slightly glossy, lower side is not glossy, shape ligulate, apex acute, cross section concave to flat, margin is entire, spines on margin absent to very weak, spines on lower side midrib absent to very weak.
Basal sheath: Anthocyanin colour absent to weak, sheath coloration yellow green (RHS 147B to RHS 144C).
Inflorescence: The inflorescence (flower spike) is a panicle of length range 10 to 17 cm positioning the flowers and berries just above the foliage. Peduncle color is yellow green (RHS 144B). Flowering season is year round.
Flowers: The flower buds are 7-10 mm long and 3-4 mm wide. Bud color changes from yellow green (RHS 147B) to violet blue (RHS N89C to 93B) prior to opening. Mature flowers have 6 petals with ovate shape. Petal color is violet-blue (RHS N89D). Anther filament color is yellow (approximately RHS 11A). Flower diameter range is 9-13 mm. Petal attitude is horizontal to slightly reflexed.
Fruit: A succulent berry, shape is globose, color of unripe berry is yellow green (RHS 144C) and color of ripe berry is approximately violet blue (RHS N89A), surface texture is smooth and glossy. Seed number is usually 6, seed color is black (RHS 203A).
Environmental tolerances: ‘Indigo Bells’ has shown potential for shade tolerance. The winter hardiness of ‘Indigo Bells’ is still undergoing testing. ‘Indigo Bells’ has excellent drought tolerance as for other Dianella varieties. After severe wilting, ‘Indigo Bells’ has been noted to recover with watering. ‘Indigo Bells’ withstands wet weather conditions and higher humidity with less root rotting disease compared to other Dianella caerulea varieties.
Disease resistance and susceptibility: ‘Indigo Bells’ has excellent tolerance to pests and diseases and good resistance to root rot, which is a common problem in Dianella caerulea. These features and other characteristics of the plant are apparent from the figures.

### ‘Indigo Bells’ Compared to Other Varieties of Dianella caerulea and Dianella brevipedunculata

In comparing ‘Indigo Bells’ with the other Dianella caerulea varieties including the maternal parent, ‘Indigo Bells’ has a more compact habit with shorter plant height than Dianella caerulea, ‘DCMP01’ (known as ‘Little Becca’), and ‘DCNC0’ (known as ‘Becca’). ‘Indigo Bells’ has a shorter flower spike height (range 10-17 cm) positioned just above the foliage compared to Dianella caerulea (range 40-90 cm), ‘DCMP01’ (range 50-60 cm) and ‘DCNC0’ (range 80-90 cm) which has the flowers positioned well above the foliage. Dianella caerulea, ‘DCMP01’ and ‘DCNC0’ flower seasonally in spring to early summer whereas ‘Indigo Bells’ has year round flowering. ‘Indigo Bells’ has a violet blue flower color (RHS N89D) whereas Dianella caerulea (40-90 cm), ‘DCMP01’ has a violet blue flower color (RHS 92A-B) and ‘DCNC0’ has a violet blue flower color (RHS 97C).

That which is claimed is:

1. A new and distinct variety of Dianella caerulea plant named ‘Indigo Bells’, substantially as described and illustrated herein.
